(19) United States
(12) Patent Application Publication
(10) Pub. No.: US 2023/0042316 A1
(43) Pub. Date: Feb. 9, 2023

(54) MONOCLONAL ANTIBODIES THAT TARGET HUMAN CD47 PROTEIN

(71) Applicant: ABVISION, INC., Milpitas, CA (US)

(72) Inventors: Chun-Jen LIN, Milpitas, CA (US); Cheng-Chi CHAO, Milpitas, CA (US); Chang-Hsin CHEN, San Jose, CA (US); Gloria ZHANG, Reno, NV (US); Guochen YAN, Reno, NV (US)

(21) Appl. No.: 17/778,636

(22) PCT Filed: Nov. 20, 2020
(86) PCT No.: PCT/US20/61677

§ 371 Date: May 20, 2022

Related U.S. Application Data
(60) Provisional application No. 62/938,311, filed on Nov. 20, 2019.

(51) Int. Cl. C07K 16/28; A61P 35/00

(52) U.S. Cl. CPC C07K 16/2803; A61P 35/00; C07K 2317/565; C07K 2317/55; C07K 2317/54; C07K 2317/31; A61K 2039/505

(57) ABSTRACT

The present disclosure provides isolated monoclonal antibodies or antigen-binding portions thereof that specifically bind to CD47 preferably human CD47 with high affinity, and can enhance tumor-targeting immune response by therapeutically boosting the phagocytic function of macrophage for cancer treatment. The disclosure also provides antibodies that are chimeric, humanized, bispecific, derivatized, single chain antibodies or portions of fusion proteins. Nucleic acid molecules encoding the antibodies of the disclosed invention and hybridoma are also provided. Pharmaceutical compositions comprising the antibodies of the disclosed invention are also provided. This disclosure also provides methods for regulating innate immune responses, as well as methods for treating cancer using an anti-CD47 antagonist antibody of the disclosed invention.

### CROSS REFERENCE TO RELATED APPLICATION

This application claims priority to U.S. Provisional Application No. 62/938,311, filed Nov. 20, 2019, the disclosure of which is incorporate by reference herein in its entirety.

### INCORPORATION OF SEQUENCE LISTING

This application includes a Sequence Listing which is being submitted in ASCII format via EFS-Web, named “AVI105PCT_ST25.txt,” which is 28 KB in size and created on Nov. 20, 2020. The contents of the Sequence Listing are incorporated herein by reference in their entirety.

### BACKGROUND

Most cancer immunotherapies available today are mainly focused on targeting the adaptive immune response. However, the components of innate immune system such as macrophages and myeloid immune cells also offer great lucrative alternatives for cancer treatment.

CD47 is a 50 kDa multipass transmembrane protein that acts as a ‘marker of self’ and is widely expressed on cell surface (also called integrin-associated protein). It interacts with the myeloid inhibitory immuno receptor SIRP alpha (also termed CD172a or SHPS-1). This interaction of SIRP alpha with CD47 controls the effector functions of innate immune cells such as host cell phagocytosis (Steven E. Kauder et al, 2018). CD47 expression and/or activity is associated with a number of diseases and disorders. Accordingly, there exists a need for therapies that target CD47, as well as better methods for making such therapies.

The CD47-SIRP-alpha interaction sends a “don't eat me” signal to the phagocytic cells. Therefore, blocking the CD47-SIRP-alpha interaction with a monoclonal antibody can provide an effective anti-cancerous treatment, i.e., increasing phagocytosis of CD47-expressing cells by macrophages (reviewed in Chao, et al, 2012 Curr Opin Immunol, 24(2): 225-32), for improved uptake and removal of cancer cells by the host's immune system. This mechanism is effective in leukemias, lymphomas, and many other types of solid tumors. Furthermore, these CD47-blocking antibodies have been shown to synergize with other therapeutic antibodies including Rituxan® and Herceptin® in tumor models. The macrophage checkpoint inhibitor 5F9 (anti-CD47 antibody) combined with rituximab showed good activity in patients with aggressive and indolent lymphoma (Advani R et al, 2018).

Various studies have found that anti CD47 antibodies cause platelet aggregation and hemagglutination of the red blood cells. When anti-CD47 antibodies interacts with the cells expressing the CD47 protein on their surface, the cells tends to aggregate, such an interaction is called as homotypic interaction. The CD47 antibody, B6H12, has been reported by Dorahy et al, 1997, to cause direct platelet aggregation in some of the target subjects. Similarly Uno S, Kinoshita Y, Azuma Y, et al, 2007, has been reported to cause hemagglutination of erythrocytes. Thus, the recognition of the non cancerous self cells by the anti-CD47 antibody is the major drawback of this therapy which needs to be addressed in the future therapeutic approaches.

The current clinical approved immunotherapies targeting CD47 have shown promising clinical results. However, the response rate of patients to these approved agents still requires improvement. Hence, there is a need in the art to identify a highly efficient anti-CD47 antibody which can effectively act upon its target with minimum hemagglutination and platelet aggregation when used alone or as combinational therapeutic agents with other drugs treatment regimens for any given combination.

### SUMMARY OF THE INVENTION

The present disclosure provides isolated monoclonal antibodies, or antigen-binding portions thereof, that specifically bind to human CD47 and inhibits its interaction with SIRP-alpha (signal regulatory protein) and thereby contributes to innate immunity.

According to an aspect, the invention provides an isolated monoclonal antibody, or antigen binding portion thereof comprising: (a) a heavy chain variable region CDR1 comprising SEQ ID NO:3; (b) a heavy chain variable region CDR2 comprising SEQ ID NO:4; (c) a heavy chain variable region CDR3 comprising SEQ ID NO:5; (d) a light chain variable region CDR1 comprising SEQ ID NO:6; (e) a light chain variable region CDR2 comprising SEQ ID NO:7 and (f) a light chain variable region CDR3 comprising SEQ ID NO:8; wherein said antibody or portion specifically binds to human CD47 and inhibits its interaction with SIRP-alpha (signal regulatory protein), thereby contributing to phagocytic function of macrophages of innate immunity.

According to another aspect, the invention provides an isolated monoclonal antibody, or antigen binding portion thereof comprising: (a) a heavy chain variable region CDR1 comprising SEQ ID NO:19; (b) a heavy chain variable region CDR2 comprising SEQ ID NO: 20; (c) a heavy chain variable region CDR3 comprising SEQ ID NO:21; (d) a light chain variable region CDR1 comprising SEQ ID NO:22; (e) a light chain variable region CDR2 comprising SEQ ID NO:23 and (f) a light chain variable region CDR3 comprising SEQ ID NO:24; wherein said antibody or portion specifically binds to human CD47 and inhibits its interaction with SIRP-alpha (signal regulatory protein), thereby contributing to phagocytic function of macrophages of innate immunity.

According to yet another aspect, the invention provides an isolated monoclonal antibody, or antigen binding portion thereof comprising: (a) a heavy chain variable region CDR1 comprising SEQ ID NO:35; (b) a heavy chain variable region CDR2 comprising SEQ ID NO:36; (c) a heavy chain variable region CDR3 comprising SEQ ID NO:37; (d) a light chain variable region CDR1 comprising SEQ ID NO:38; (e) a light chain variable region CDR2 comprising SEQ ID NO:39 and (f) a light chain variable region CDR3 comprising SEQ ID NO:40; wherein said antibody or portion specifically binds to human CD47 and inhibits its interaction with SIRP-alpha (signal regulatory protein), thereby contributing to phagocytic function of macrophages of innate immunity.

According to yet another aspect, the invention provides an isolated monoclonal antibody, or antigen binding portion thereof comprising: (a) a heavy chain variable region CDR1 comprising SEQ ID NO:51; (b) a heavy chain variable region CDR2 comprising SEQ ID NO:52; (c) a heavy chain variable region CDR3 comprising SEQ ID NO:53; (d) a light chain variable region CDR1 comprising SEQ ID NO:54; (e) a light chain variable region CDR2 comprising SEQ ID NO:55 and (f) a light chain variable region CDR3 comprising SEQ ID NO:56; wherein said antibody or portion specifically binds to human CD47 and inhibits its interaction with SIRP-alpha (signal regulatory protein), thereby contributing to phagocytic function of macrophages of innate immunity.

According to yet another aspect, the invention provides an isolated monoclonal antibody, or antigen binding portion thereof comprising: (a) a heavy chain variable region CDR1 comprising SEQ ID NO:67; (b) a heavy chain variable region CDR2 comprising SEQ ID NO:68; (c) a heavy chain variable region CDR3 comprising SEQ ID NO:69; (d) a light chain variable region CDR1 comprising SEQ ID NO:70; (e) a light chain variable region CDR2 comprising SEQ ID NO:71 and (f) a light chain variable region CDR3 comprising SEQ ID NO:72; wherein said antibody or portion specifically binds to human CD47 and inhibits its interaction with SIRP-alpha (signal regulatory protein), thereby contributing to phagocytic function of macrophages of innate immunity.

In some embodiments, the monoclonal antibody, or said antigen-binding portion thereof stimulates an anti-tumor immune response. In some embodiments, the monoclonal antibody can be a chimeric antibody or a humanized antibody. In some embodiments, the anti-CD47 antibodies inhibits CD47 protein interaction with SIRP-alpha (signal regulatory protein) thereby contributing to phagocytic function of macrophages of innate immunity.

According to another aspect, the invention pertains to an isolated anti-CD47 monoclonal antibody, or antigen-binding portion thereof, comprising: (a) a heavy chain variable region comprising an amino acid sequence selected from the group consisting of SEQ ID NOs: 1, 3, 4 and 5; and (b) a light chain variable region comprising an amino acid sequence selected from the group consisting of SEQ ID NOs: 2, 6, 7 and 8.

According to another aspect, the invention pertains to an isolated anti-CD47 monoclonal antibody, or antigen-binding portion thereof, comprising: (a) a heavy chain variable region comprising an amino acid sequence selected from the group consisting of SEQ ID NOs: 17, 19, 20 and 21; and (b) a light chain variable region comprising an amino acid sequence selected from the group consisting of SEQ ID NOs: 18, 22, 23 and 24.

According to another aspect, the invention pertains to an isolated anti-CD47 monoclonal antibody, or antigen-binding portion thereof, comprising: (a) a heavy chain variable region comprising an amino acid sequence selected from the group consisting of SEQ ID NOs: 33, 35, 36 and 37; and (b) a light chain variable region comprising an amino acid sequence selected from the group consisting of SEQ ID NOs: 34, 38, 39 and 40.

According to another aspect, the invention pertains to an isolated anti-CD47 monoclonal antibody, or antigen-binding portion thereof, comprising: (a) a heavy chain variable region comprising an amino acid sequence selected from the group consisting of SEQ ID NOs: 49, 51, 52 and 53; and (b) a light chain variable region comprising an amino acid sequence selected from the group consisting of SEQ ID NOs: 50, 54, 55 and 56.

According to another aspect, the invention pertains to an isolated anti-CD47 monoclonal antibody, or antigen-binding portion thereof, comprising: (a) a heavy chain variable region comprising an amino acid sequence selected from the group consisting of SEQ ID NOs: 65, 67, 68 and 69; and (b) a light chain variable region comprising an amino acid sequence selected from the group consisting of SEQ ID NOs: 66, 70, 71 and 72.

In a preferred embodiment, an isolated monoclonal antibody or an antigen-binding portion thereof that specifically binds to human CD47, wherein said antibody comprises: a heavy chain variable domain selected from SEQ ID NO: 1, 17, 33, 49, or 65 and a light chain variable domain selected from SEQ ID NO: 2, 18, 34, 50 or 66. In some embodiments, the monoclonal antibody, or an antigen-binding portion thereof stimulates an anti-tumor immune response.

According to yet another aspect, the antibodies of the invention can be, for example, full-length antibodies, for example of an IgG1, IgG2, IgG3, or IgG4 isotype. Alternatively, the disclosed antibodies can be antibody fragments, such as Fab, Fab′ and F(ab′)₂ fragments, diabody, triabody, tetrabody, single-chain variable region fragment (scFv), disulfide-stabilized variable region fragment (dsFv), and half antibodies.

The antibodies of the disclosed invention can be further engineered into formats suitable for human therapeutics by modifications that minimize immunogenicity. Suitable antibodies include, but are not limited to chimeric antibodies and humanized antibodies. The affinity, stability and specificity of the disclosed antibodies can also be further optimized by techniques known to one of skill in the art such as light-chain shuffling. Other formats can involve oligomerization (multivalent), drug conjugation, bispecific antibody and fusion of the disclosed antibodies with other functional proteins.

In yet another aspect, the invention provides a bispecific antibody comprising an antibody or portion binding to PD-1, CTLA-4 or other immune checkpoint targets, cancer-related targets, or immune-related disease targets and the antibody or portion binding to CD47. In another aspect, a bispecific antibody comprising an antibody or portion binding to OX40.

In yet another aspect, a pharmaceutical composition comprising an isolated monoclonal antibody, or antigen-binding portion thereof and a pharmaceutically acceptable carrier are also provided.

In yet another aspect, the invention provides method of enhancing an immune response using the anti-CD47 antibodies of the disclosed invention. For example, in one embodiment, the disclosed invention provides a method for treating a subject in need thereof, wherein said response is indicated by activating tumor-specific effector and memory T-cells and enhancing tumor-targeting immune response, comprising the step of administering to the subject an effective amount of the antibody or antigen-binding portion of the disclosed invention.

In yet another aspect, the invention provides a method for treating cancer in a human comprising the step of administering to the human the antibody or antigen-binding portion of the disclosed invention in an amount effective to treat said cancer and infectious diseases.

In yet another aspect, the invention provides a monoclonal antibody or an antigen-binding portion thereof that specifically binds to human CD47, wherein said antibody comprises: a heavy chain variable domain amino acid sequence encoded by a nucleic acid sequence comprising SEQ ID NOs: 9, 11, 12 and 13; and a light chain variable domain amino acid sequence encoded by a nucleic acid sequence comprising SEQ ID NOs: 10, 14, 15 and 16. In yet another aspect, the invention also provides nucleic acid molecules encoding the heavy and/or light chain, or antigen-binding portions thereof, of an anti-CD47 antibody.

In yet another aspect, the invention provides a monoclonal antibody or an antigen-binding portion thereof that specifically binds to human CD47, wherein said antibody comprises: a heavy chain variable domain amino acid sequence encoded by a nucleic acid sequence comprising SEQ ID NOs: 25, 27, 28 and 29; and a light chain variable domain amino acid sequence encoded by a nucleic acid sequence comprising SEQ ID NOs: 26, 30, 31 and 32.

In yet another aspect, the invention provides a monoclonal antibody or an antigen-binding portion thereof that specifically binds to human CD47, wherein said antibody comprises: a heavy chain variable domain amino acid sequence encoded by a nucleic acid sequence comprising SEQ ID NOs: 41, 43, 44 and 45; and a light chain variable domain amino acid sequence encoded by a nucleic acid sequence comprising SEQ ID NOs: 42, 46, 47 and 48.

In yet another aspect, the invention provides a monoclonal antibody or an antigen-binding portion thereof that specifically binds to human CD47, wherein said antibody comprises: a heavy chain variable domain amino acid sequence encoded by a nucleic acid sequence comprising SEQ ID NOs: 57, 59, 60 and 61; and a light chain variable domain amino acid sequence encoded by a nucleic acid sequence comprising SEQ ID NOs: 58, 62, 63 and 64.

In yet another aspect, the invention provides a monoclonal antibody or an antigen-binding portion thereof that specifically binds to human CD47, wherein said antibody comprises: a heavy chain variable domain amino acid sequence encoded by a nucleic acid sequence comprising SEQ ID NOs: 73, 75, 76 and 77; and a light chain variable domain amino acid sequence encoded by a nucleic acid sequence comprising SEQ ID NOs: 74, 78, 79 and 80.

Other features and advantages of the instant disclosure will be apparent from the following detailed description and examples, which should not be construed as limiting. The contents of all references, GenBank entries, patents and published patent applications cited throughout this application are expressly incorporated herein by reference.

### BRIEF DESCRIPTION OF THE DRAWINGS

FIG. 1 shows binding assay results for certain anti-human CD47 antibodies of the present invention.

FIG. 2 shows binding assay results for certain anti-human CD47 antibodies of the present invention.

FIG. 3 shows binding assay results for certain anti-human CD47 antibodies of the present invention.

FIG. 4 shows phagocytosis assay results for certain anti-human CD47 antibodies of the present invention.

FIG. 5 shows phagocytosis assay results for certain anti-human CD47 antibody of the present invention.

FIG. 6 shows binding assay results for certain anti-human CD47 antibodies of the present invention.

FIG. 7 shows in vivo anti-tumor efficacy anti-human CD47 antibodies of the present invention.

### DETAILED DESCRIPTION OF THE INVENTION

The following embodiment and aspects thereof are described and illustrated in conjunction with systems, compositions and methods which are meant to be exemplary and illustrative, not limiting in scope.

The present disclosure relates to an isolated monoclonal antibody that inhibits CD47 signaling and contributes to the enhancement of the innate immunity. In certain embodiments, the antibodies of the disclosed invention are derived from identified heavy and light chain germline sequences and/or comprise identified structural features such as CDR regions comprising identified amino acid sequences. This disclosure provides isolated antibodies, methods of making such antibodies and antigen-binding portions thereof of the disclosed invention. The invention also relates to methods of using the antibodies, such as using the antagonistic CD47 antibodies of the disclosed invention to enhance the tumor targeting immune responses, alone or in combination with other immunostimulatory antibodies. The antibody according to the invention can also be used in various other modified formats, wherein the modification can be by oligomerization, drug conjugation, bi-specific antibodies and the fusion with other functional proteins suitable for human therapeutics that minimize immunogenicity, maximize affinity, stability and specificity. Accordingly, also provided are methods of using the antagonistic CD47 antibodies of the disclosed invention for example, including but not limited to, treating cancer in a human.

The term “epitope” as used herein can include any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the equilibrium dissociation constant is ≤1 μM, preferably ≤100 nM, more preferably ≤10 nM and most preferably ≤1 nM.

The term “immune response” as used herein can refer to the action of or activation of, for example, lymphocytes, antigen presenting cells, phagocytic cells, granulocytes, and soluble macromolecules or small organic molecules such as metabolites produced by the above cells or the liver (including antibodies, cytokines, and complement), components of innate immune system that results in selective damage to, destruction of, blocking of, or elimination from an organism of invading pathogens, cells or tissues infected with pathogens, interaction within molecules, cancerous cells, or, in cases of autoimmunity or pathological inflammation, normal organismal cells or tissues. The immune response, as used herein, refers to the interaction between anti CD47 monoclonal antibodies that specifically bind on human CD47 protein and neutralize the interaction between CD47 and SIRP (signal regulatory protein) to therapeutically boost the phagocytic function of macrophage for cancer treatment.

The term “antibody” as used herein can include whole antibodies, F(ab′)2 fragment, diabody, triabody, tetrabody, bispecific antibody, monomeric antibodies and any antigen binding fragment (i.e., “antigen-binding portion”) or single-chain variable region fragment (scFv), or disulfide-stabilized variable region fragment (dsFv) thereof. Whole antibodies are glycoproteins comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region (CL). The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, can refer to one or more fragments of an antibody that retain the ability to specifically bind to an antigen (e.g., a CTLA-4 protein). It has been shown that the antigen-binding function of an antibody can be performed by fragments of a full-length antibody. Examples of binding fragments encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3.sup.rd ed. 1993); (iv) a Fd fragment consisting of the VH and CH1 domains; (v) a Fv fragment consisting of the VL and VH domains of a single arm of an antibody, (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH are encoded by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as intact antibodies.

An “isolated antibody”, as used herein, can refer to an antibody that is substantially free of other antibodies having different antigenic specificities (e.g., an isolated antibody that specifically binds a CD47 protein can be substantially free of antibodies that specifically bind antigens other than CD47 proteins). An isolated antibody that specifically binds a human CD47 protein can, however, have cross-reactivity to other antigens, such as CD47 proteins from other species. Moreover, an isolated antibody can be substantially free of other cellular material and/or chemicals.

Anti-CD47 antagonistic antibody-producing cells, e.g., hybridomas, can be selected, cloned and further screened for desirable characteristics, including robust growth, high antibody production and desirable antibody characteristics, as further discussed below. In a preferred embodiment, the anti-CD47 antibodies were created by electrofusion of human CD47-immunized mouse spleenocytes (Balb/c strain) with SP2/0-Ag14 cells (ATCC). Splenocytes were collected from balb/c mice hyperimmunized with purchased recombinant human CD47 protein. Fused cells were seeded into 96-well plates and cultured medium was screened for binding with antigen-coated magnetic beads. Positive wells were further expanded and follow a limited dilution to isolate monoclonal hybridomas. Purified antibodies were used to test their ability to bind CD47 and to neutralize interaction with SIRP. The terms “monoclonal antibody” or “monoclonal antibody composition” as used herein can refer to a preparation of antibody molecules of single molecular composition. A monoclonal antibody composition displays a single binding specificity and affinity for a particular epitope. The monoclonal antibodies herein are developed in the forms of humanized biologics, bispecific antibodies and antibody-fusion proteins.

The term “recombinant human antibody”, as used herein, can refer to all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” can refer to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. Additional framework region modifications can be made within the human framework sequences. In another embodiment, the term “humanized antibody” can refer to antibodies in which CDR sequences derived from the germline of another mammalian species, such as a mouse, have been grafted onto human framework sequences. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” can refer to antibodies in which the variable region sequences can be derived from one species and the constant region sequences can be derived from another species, such as an antibody in which the variable region sequences can be derived from a mouse antibody and the constant region sequences can be derived from a human antibody.

As used herein, an antibody that “specifically binds human CD47” can refer to an antibody that binds to a human CD47 protein (and possibly a CD47 protein from one or more non-human species) and can enhance tumor-targeting immune response by activating tumor-specific innate immune response. Antagonistic CD47 antibody can potentially serve as single therapy or in combination with other immune checkpoint therapies. Preferably, the antibody binds to a human CD47 protein with “high affinity,” namely with a Kd of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less.

The term “high affinity” for an IgG antibody can refer to an antibody having an EC₅₀ of 1×10⁻⁶ M or less, more preferably 1×10⁻⁷ M or less, even more preferably 1×10⁻⁸ M or less, even more preferably 1×10⁻⁹ M or less, even more preferably 1×10⁻¹° M or less for a target antigen. However, “high affinity” binding can vary for other antibody isotypes.

As used herein, the term “inhibit” refers to any decrease in, for example a particular action, function, or interaction. For example, a biological function, such as the function of a protein and/or binding of one protein to another, is inhibited if it is decreased as compared to a reference state, such as a control like a wild-type state or a state in the absence of an applied agent. For example, the binding of a CD47 protein to one or more of its ligands, such as, and/or resulting CD47 signaling and immune effects is decreased, if the binding, signaling, and other immune effects are decreased due to contact with an agent, such as an anti-CD47 antibody, in comparison to when the CD47 protein is not contacted with the agent. Such inhibition or deficiency can be induced, such as by application of agent at a particular time and/or place, or can be constitutive, such as by continual administration. Such inhibition or deficiency can also be partial or complete (e.g., essentially no measurable activity in comparison to a reference state, such as a control like a wild-type state). Essentially complete inhibition or deficiency is referred to as blocked.

The term “subject” can refer to any human or non-human animal. The term “nonhuman animal” includes all vertebrates, e.g., mammals and non-mammals, such as nonhuman primates, sheep, dogs, cats, cows, horses, chickens, rabbits, mice, rats, amphibians, and reptiles, although mammals are preferred, such as non-human primates, sheep, dogs, cats, cows and horses.

The binding of an antibody of the disclosed invention to CD47 can be assessed using one or more techniques well established in the art. For example, in a preferred embodiment, the purified anti-CD47 antibody can be tested by various biochemical assays such as ELISA assays, for example by assessment of the binding with recombinant CD47 protein. Still other suitable binding assays include but are not limited to a flow cytometry assay in which the antibody is reacted with a cell line that expresses human CD47, such as Jurkat cells that have been transfected to express CD47 protein (e.g., human CD47) on their cell surface. Additionally, the binding of the antibody, including the binding kinetics (e.g., K_{D} value) can be tested in Biacore binding assays and the like.

Preferably, an antibody of the disclosed invention binds to a CD47 protein with an EC₅₀ of 5×10⁻⁸ M or less, binds to a CD47 protein with a EC₅₀ of 2×10⁻⁸ M or less, binds to a CD47 protein with a EC₅₀ of 5×10⁻⁹ M or less, binds to a CD47 protein with a EC₅₀ of 4×10⁻⁹ M or less, binds to a CD47 protein with a EC₅₀ of 3×10⁻⁹ M or less, binds to a CD47 protein with a EC₅₀ of 2×10⁻⁹ M or less, binds to a CD47 protein with a EC₅₀ of 1×10⁻⁹ M or less.

B cells or hybridoma expressing the antibodies against the antigen can be isolated and the nucleic acid sequences encoding the heavy chain variable domain (VH) and light chain variable domain (VL) can be obtained by next generation sequencing. The encoded amino acid sequences of VH and VL can be obtained from the nucleic acid sequences. These techniques are well known by the person of ordinary skill in the art.

The VH amino acid sequence of Clone #6B8 is shown in SEQ ID NO: 1. The VL amino acid sequence of Clone #6B8 is shown in SEQ ID NO:2. The VH amino acid sequence of Clone #6E12 is shown in SEQ ID NO:17. The VL amino acid sequence of Clone #6E12 is shown in SEQ ID NO:18. The VH amino acid sequence of Clone #6G7 is shown in SEQ ID NO:33. The VL amino acid sequence of Clone #6G7 is shown in SEQ ID NO:34. The VH amino acid sequence of Clone #6G10 is shown in SEQ ID NO:49. The VL amino acid sequence of Clone #6G10 is shown in SEQ ID NO:50. The VH amino acid sequence of Clone #1G4 is shown in SEQ ID NO:65. The VL amino acid sequence of Clone #1G4 is shown in SEQ ID NO:66.

Accordingly, in one aspect, this disclosure provides an isolated monoclonal antibody, or antigen-binding portion thereof comprising: (a) a heavy chain variable region comprising an amino acid sequence comprising SEQ ID NO:1; and (b) a light chain variable region comprising an amino acid sequence comprising SEQ ID NO:2; wherein the antibody specifically binds to human CD47.

In another aspect, this disclosure provides an isolated monoclonal antibody, or antigen-binding portion thereof comprising: (a) a heavy chain variable region comprising an amino acid sequence comprising SEQ ID NO:17; and (b) a light chain variable region comprising an amino acid sequence comprising SEQ ID NO:18; wherein the antibody specifically binds to human CD47.

In another aspect, this disclosure provides an isolated monoclonal antibody, or antigen-binding portion thereof comprising: (a) a heavy chain variable region comprising an amino acid sequence comprising SEQ ID NO:33; and (b) a light chain variable region comprising an amino acid sequence comprising SEQ ID NO:34; wherein the antibody specifically binds to human CD47.

In another aspect, this disclosure provides an isolated monoclonal antibody, or antigen-binding portion thereof comprising: (a) a heavy chain variable region comprising an amino acid sequence comprising SEQ ID NO:49; and (b) a light chain variable region comprising an amino acid sequence comprising SEQ ID NO:50; wherein the antibody specifically binds to human CD47.

In another aspect, this disclosure provides an isolated monoclonal antibody, or antigen-binding portion thereof comprising: (a) a heavy chain variable region comprising an amino acid sequence comprising SEQ ID NO:65; and (b) a light chain variable region comprising an amino acid sequence comprising SEQ ID NO:66; wherein the antibody specifically binds to human CD47.

In some embodiments, the anti-CD47 monoclonal antibody, or an antigen-binding portion thereof stimulates an anti-tumor immune response. In some embodiments, the anti-CD47 monoclonal antibody can be a bispecific, antibody-fusion protein, immunoconjugate, immunotoxins and/or chimeric antibody.

In another aspect, this disclosure provides antibodies that comprise the heavy chain and light chain CDR1, CDR2 and CDR3 of Clone #6B8. The amino acid sequence of the VH CDR1 of Clone #6B8 is shown in SEQ ID NO:3. The amino acid sequence of the VH CDR2 of Clone #6B8 is shown in SEQ ID NO:4. The amino acid sequence of the VH CDR3 of Clone #6B8 is shown in SEQ ID NO:5. The amino acid sequences of the VL CDR1 of Clone #6B8 is shown in SEQ ID NO:6. The amino acid sequences of the VL CDR2 of Clone #6B8 is Lys-Ile-Ser, shown in SEQ ID NO:7. The amino acid sequences of the VL CDR3 of Clone #6B8 is shown in SEQ ID NO:8.

In another aspect, this disclosure provides antibodies that comprise the heavy chain and light chain CDR1, CDR2 and CDR3 of Clone #6E12. The amino acid sequence of the VH CDR1 of Clone #6E12 is shown in SEQ ID NO:19. The amino acid sequence of the VH CDR2 of Clone #6E12 is shown in SEQ ID NO:20. The amino acid sequence of the VH CDR3 of Clone #6E12 is shown in SEQ ID NO:21. The amino acid sequences of the VL CDR1 of Clone #6E12 is shown in SEQ ID NO:22. The amino acid sequences of the VL CDR2 of Clone #6E12 is Ser-Ala-Asn, shown in SEQ ID NO:23. The amino acid sequences of the VL CDR3 of Clone #6E12 is shown in SEQ ID NO:24.

In another aspect, this disclosure provides antibodies that comprise the heavy chain and light chain CDR1, CDR2 and CDR3 of Clone #6G7. The amino acid sequence of the VH CDR1 of Clone #6G7 is shown in SEQ ID NO:35. The amino acid sequence of the VH CDR2 of Clone #6G7 is shown in SEQ ID NO:36. The amino acid sequence of the VH CDR3 of Clone #6G7 is shown in SEQ ID NO:37. The amino acid sequences of the VL CDR1 of Clone #6G7 is shown in SEQ ID NO:38. The amino acid sequences of the VL CDR2 of Clone #6G7 is Arg-Val-Asn, shown in SEQ ID NO:39. The amino acid sequences of the VL CDR3 of Clone #6G7 is shown in SEQ ID NO:40.

In another aspect, this disclosure provides antibodies that comprise the heavy chain and light chain CDR1, CDR2 and CDR3 of Clone #6G10. The amino acid sequence of the VH CDR1 of Clone #6G10 is shown in SEQ ID NO:51. The amino acid sequence of the VH CDR2 of Clone #6G10 is shown in SEQ ID NO:52. The amino acid sequence of the VH CDR3 of Clone #6G10 is shown in SEQ ID NO:53. The amino acid sequences of the VL CDR1 of Clone #6G10 is shown in SEQ ID NO:54. The amino acid sequences of the VL CDR2 of Clone #6G10 is Lys-Val-Ser, shown in SEQ ID NO:55. The amino acid sequences of the VL CDR3 of Clone #6G10 is shown in SEQ ID NO:56.

In another aspect, this disclosure provides antibodies that comprise the heavy chain and light chain CDR1, CDR2 and CDR3 of Clone #1G4. The amino acid sequence of the VH CDR1 of Clone #1G4 is shown in SEQ ID NO:67. The amino acid sequence of the VH CDR2 of Clone #1G4 is shown in SEQ ID NO:68. The amino acid sequence of the VH CDR3 of Clone #1G4 is shown in SEQ ID NO:69. The amino acid sequences of the VL CDR1 of Clone #1G4 is shown in SEQ ID NO:70. The amino acid sequences of the VL CDR2 of Clone #1G4 shown in SEQ ID NO:71. The amino acid sequences of the VL CDR3 of Clone #1G4 is shown in SEQ ID NO:72.

The CDR regions can be delineated using the Kabat system (Kabat et al. (1991) Sequences of Proteins of Immunological Interest, Fifth Edition, U.S. Department of Health and Human Services, NIH Publication No. 91-3242).

In another aspect, this disclosure provides amino acid sequences encoding the heavy chain and light chain variable domains of the monoclonal antibody Clone #6B8 (Table 1).

In another aspect, this disclosure provides amino acid sequences encoding the heavy chain and light chain variable domains of the monoclonal antibody Clone #6E12 (Table 1).

In another aspect, this disclosure provides amino acid sequences encoding the heavy chain and light chain variable domains of the monoclonal antibody Clone #6G7 (Table 1).

In another aspect, this disclosure provides amino acid sequences encoding the heavy chain and light chain variable domains of the monoclonal antibody Clone #6G10 (Table 1).

In another aspect, this disclosure provides amino acid sequences encoding the heavy chain and light chain variable domains of the monoclonal antibody Clone #1G4 (Table 1).

In another aspect, this disclosure provides polynucleotide sequences encoding the heavy chain and light chain variable domains of the monoclonal antibody Clone #6B8 (Table 2).

In another aspect, this disclosure provides polynucleotide sequences encoding the heavy chain and light chain variable domains of the monoclonal antibody Clone #6E12 (Table 2).

In another aspect, this disclosure provides polynucleotide sequences encoding the heavy chain and light chain variable domains of the monoclonal antibody Clone #6G7 (Table 2).

In another aspect, this disclosure provides polynucleotide sequences encoding the heavy chain and light chain variable domains of the monoclonal antibody Clone #6G10 (Table 2).

In another aspect, this disclosure provides polynucleotide sequences encoding the heavy chain and light chain variable domains of the monoclonal antibody Clone #1G4 (Table 2).

Antibodies can be affinity maturated by light-chain shuffling combined with or without random mutagenesis of its heavy chain variable domain and panning against CD47. The VL CDR1, CDR2 and CDR3 of the antibodies mentioned in this disclosed invention can be optimized with light-chain shuffling to create other CD47 binding molecules of the disclosed invention.

An antibody of the disclosed invention further can be prepared using an antibody having one or more of the VH and/or VL sequences disclosed herein as starting material to engineer a modified antibody, which modified antibody can have altered properties from the starting antibody. An antibody can be engineered by modifying one or more residues within one or both variable regions (i.e., VH and/or VL), for example within one or more CDR regions and/or within one or more framework regions. Additionally, or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs can be more diverse between individual antibodies than sequences outside of CDRs.

Because CDR sequences can be responsible for most antibody-antigen interactions, it can be possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al. (1998) Nature 332:323-327; Jones et al. (1986) Nature 321: 522-525; Queen et al. (1989) Proc. Natl. Acad. See. U.S.A. 86: 10029-10033; U.S. Pat. Nos. 5,225,539; 5,530,101; 5,585,089; 5,693,762 and 6,180,370.)

Accordingly, another embodiment of the disclosed invention pertains to an isolated monoclonal antibody, or antigen-binding portion thereof, comprising a heavy chain variable region comprising CDR1, CDR2, and CDR3 sequences comprising an amino acid sequence of SEQ ID NO:3, SEQ ID NO:4, and SEQ ID NO:5, respectively, and a light chain variable region a comprising CDR1, CDR2, and CDR3 sequences comprising an amino acid sequence of SEQ ID NO:6, Lys-Ile-Ser SEQ ID NO:7, and SEQ ID NO:8, respectively.

Accordingly, another embodiment of the disclosed invention pertains to an isolated monoclonal antibody, or antigen-binding portion thereof, comprising a heavy chain variable region comprising CDR1, CDR2, and CDR3 sequences comprising an amino acid sequence of SEQ ID NO:19, SEQ ID NO:20, and SEQ ID NO:21, respectively, and a light chain variable region a comprising CDR1, CDR2, and CDR3 sequences comprising an amino acid sequence of SEQ ID NO:22, Ser-Ala-Asn SEQ ID NO:23, and SEQ ID NO:24, respectively.

Accordingly, another embodiment of the disclosed invention pertains to an isolated monoclonal antibody, or antigen-binding portion thereof, comprising a heavy chain variable region comprising CDR1, CDR2, and CDR3 sequences comprising an amino acid sequence of SEQ ID NO:35, SEQ ID NO:36, and SEQ ID NO:37, respectively, and a light chain variable region a comprising CDR1, CDR2, and CDR3 sequences comprising an amino acid sequence of SEQ ID NO:38, Arg-Val-Asn SEQ ID NO:39, and SEQ ID NO:40, respectively.

Accordingly, another embodiment of the disclosed invention pertains to an isolated monoclonal antibody, or antigen-binding portion thereof, comprising a heavy chain variable region comprising CDR1, CDR2, and CDR3 sequences comprising an amino acid sequence of SEQ ID NO:51, SEQ ID NO:52, and SEQ ID NO:53, respectively, and a light chain variable region a comprising CDR1, CDR2, and CDR3 sequences comprising an amino acid sequence of SEQ ID NO:54, Lys-Val-Ser SEQ ID NO:55, and SEQ ID NO:56, respectively.

Accordingly, another embodiment of the disclosed invention pertains to an isolated monoclonal antibody, or antigen-binding portion thereof, comprising a heavy chain variable region comprising CDR1, CDR2, and CDR3 sequences comprising an amino acid sequence of SEQ ID NO:67, SEQ ID NO:68, and SEQ ID NO:69, respectively, and a light chain variable region a comprising CDR1, CDR2, and CDR3 sequences comprising an amino acid sequence of SEQ ID NO:70, SEQ ID NO:71, and SEQ ID NO:72, respectively.

Such framework sequences can be obtained from public DNA databases or published references that include germline antibody gene sequences. For example, the CDR regions can be delineated using IMGT reference library (Lefranc, M.-P. and Lefranc, G, The Immunoglobulin Facts Book Academic Press, London, UK (2001)).

Antibody protein sequences are compared against a compiled protein sequence database using one of the sequence similarity searching methods called the Gapped BLAST (Altschul et al. (1997), supra), which is well known to those skilled in the art. The compositions and methods of the presently disclosed invention are not limited to variants of the exemplary sequences disclosed herein but include those having at least 90%, at least 95% and at least 99% sequence identity to an exemplary sequence disclosed herein.

Given that each of these antibodies Fab, can bind to human CD47, the VH and VL sequences can be “mixed and matched” to create other anti-CD47 binding molecules of the invention. Preferably, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence.

Accordingly, in one aspect, this disclosure provides an isolated monoclonal antibody, or antigen-binding portion thereof comprising: (a) a heavy chain variable region comprising an amino acid sequence of SEQ ID NO 1; and (b) a light chain variable region comprising an amino acid sequence of SEQ ID NO 2; wherein the antibody specifically binds human CD47 and inhibit the CD47 signaling that contribute towards enhancing the innate immunity.

Accordingly, in one aspect, this disclosure provides an isolated monoclonal antibody, or antigen-binding portion thereof comprising: (a) a heavy chain variable region comprising an amino acid sequence of SEQ ID NO 17; and (b) a light chain variable region comprising an amino acid sequence of SEQ ID NO 18; wherein the antibody specifically binds human CD47 and inhibit the CD47 signaling that contribute towards enhancing the innate immunity.

Accordingly, in one aspect, this disclosure provides an isolated monoclonal antibody, or antigen-binding portion thereof comprising: (a) a heavy chain variable region comprising an amino acid sequence of SEQ ID NO 33; and (b) a light chain variable region comprising an amino acid sequence of SEQ ID NO 34; wherein the antibody specifically binds human CD47 and inhibit the CD47 signaling that contribute towards enhancing the innate immunity.

Accordingly, in one aspect, this disclosure provides an isolated monoclonal antibody, or antigen-binding portion thereof comprising: (a) a heavy chain variable region comprising an amino acid sequence of SEQ ID NO 49; and (b) a light chain variable region comprising an amino acid sequence of SEQ ID NO 50; wherein the antibody specifically binds human CD47 and inhibit the CD47 signaling that contribute towards enhancing the innate immunity.

Accordingly, in one aspect, this disclosure provides an isolated monoclonal antibody, or antigen-binding portion thereof comprising: (a) a heavy chain variable region comprising an amino acid sequence of SEQ ID NO 65; and (b) a light chain variable region comprising an amino acid sequence of SEQ ID NO 66; wherein the antibody specifically binds human CD47 and inhibit the CD47 signaling that contribute towards enhancing the innate immunity.

A nucleic acid molecule encoding the heavy or entire light chain of an anti-CD47 antibody or portions thereof can be isolated from any source that produces such an antibody. In various embodiments, the nucleic acid molecules are isolated from a B cell isolated from an animal immunized with CD47 or from an immortalized cell derived from such a B cell that expresses an anti-CD47 antibody. Methods of isolating mRNA encoding an antibody are well-known in the art. See, e.g., Sambrook et al. The mRNA may be used to produce cDNA for use in the polymerase chain reaction (PCR) or cDNA cloning of antibody genes. In a preferred embodiment, the nucleic acid molecule is isolated from a hybridoma that has as one of its fusion partners a human immunoglobulin producing cell from a non-human transgenic animal. In another embodiment, the nucleic acid can be isolated from a non-human, non-transgenic animal. The nucleic acid molecules isolated from a non-human, nontransgenic animal may be used, e.g., for humanized antibodies.

In another aspect, the present disclosure provides a pharmaceutical composition comprising one or more antibodies of the present invention formulated together with a pharmaceutically acceptable carrier. The composition may optionally contain one or more additional pharmaceutically active ingredients, such as another antibody or a drug. The pharmaceutical compositions of the disclosed invention also can be administered in combination therapy with, for example, another immunostimulatory agent, an anti-cancer agent, an antiviral agent, or a vaccine, such that the anti-CD47 antibody enhances the immune response stimulated by the vaccine.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the anti-CD47 antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies of the present invention can be administered by a variety of methods known in the art, although for many therapeutic applications, the preferred route/mode of administration is subcutaneous, intramuscular, or intravenous infusion. As will be appreciated by the skilled artisan, the route and/or mode of administration will vary depending upon the desired results.

In certain embodiments, the antibody compositions active compound may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

In certain embodiments, an anti-CD47 antibody of the disclosed invention can be orally administered, for example, with an inert diluent or an assimilable edible carrier. The compound (and other ingredients, if desired) can also be enclosed in a hard or soft-shell gelatin capsule, compressed into tablets, or incorporated directly into the subject's diet. For oral therapeutic administration, the anti-CD47 antibodies can be incorporated with excipients and used in the form of ingestible tablets, buccal tablets, troches, lozenge, capsules, elixirs, suspensions, syrups, wafers, and the like. To administer a compound of the disclosed invention by other than parenteral administration, it may be necessary to coat the compound with, or co-administer the compound with, a material to prevent its inactivation.

Additional active compounds also can be incorporated into the disclosed compositions. In certain embodiments, an anti-CD47 antibody of the disclosed invention is co-formulated with and/or co-administered with one or more additional therapeutic agents. These agents include, without limitation, antibodies that bind other targets (e.g., antibodies that bind one or more growth factors or cytokines or their cell surface receptors, such as anti-PD-1 and anti-CTLA-4 antibody), antineoplastic agents, antitumor agents, chemotherapeutic agents, peptide analogues that activate CD40, soluble CD40L, one or more chemical agents that activates CD40, CpG oligodeoxynucleotides and/or other agents known in the art that can enhance an immune response against tumor cells, e.g., IFN-1, IL-2, IL-8, IL-12, IL-15, IL-18, IL-23, IFN-γ, and GM-CSF. Such combination therapies may require lower dosages of the anti-CD47 antagonist antibody as well as the co-administered agents, thus avoiding possible toxicities or complications associated with the various immonotherapies. The current clinical approved immunotherapies targeting immune checkpoints, such as PD-1 and CTLA-4 have shown promising clinical results. However, the response rate of patients to these approved agents is still not satisfactory. The new class of immune checkpoint targets, including CD47, can enhance tumor-targeting immune response by activating innate immune response. Antagonistic CD47 antibody can potentially serve as single therapy or in combination with other immune checkpoint therapies.

Anti-CD47 antibodies of the disclosed invention and compositions comprising them also may be administered in combination with other therapeutic regimens, in particular in combination with radiation treatment.

The pharmaceutical compositions of the disclosed invention can include pharmaceutically acceptable salts. A “pharmaceutically acceptable salt” can refer to a salt that retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the antibody can be administered as a sustained release formulation, in which case less frequent administration can be required.

In certain embodiments, antibodies can be further developed into formats suitable for human therapeutics by modifications that minimize immunogenicity and maximize affinity, stability and specificity. Other formats which might involve oligomerization, drug conjugation and the fusion with other functional proteins.

An exemplary, non-limiting range for a therapeutically or prophylactically effective amount of an antibody or antibody portion of the disclosed invention is 0.025 to 50 mg/kg, more preferably 0.1 to 50 mg/kg, more preferably 0.1 to 25 mg/kg, 0.1 to 10 mg/kg and 0.1 to 3 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Bispecific antibodies or antigen-binding fragments can be produced by a variety of methods including fusion of hybridomas or linking of Fab′ fragments. See, e.g., Songsivilai & Lachmarm, Clin. Exp. Immunol. 79: 315-321 (1990), Kostelny et al., J. Immunol. 148:1547-1553 (1992). In addition, bispecific antibodies can be formed as “diabodies” or “Janusins.” In some embodiments, the bispecific antibody binds to two different epitopes of CD47. In some embodiments, the bispecific antibody has a first heavy chain and a first light chain from monoclonal antibody Clone #6B8 (Table 1), and an additional antibody heavy chain and light chain. In some embodiments, the bispecific antibody has a first heavy chain and a first light chain from monoclonal antibody Clone #6E12, and an additional antibody heavy chain and light chain. In some embodiments, the bispecific antibody has a first heavy chain and a first light chain from monoclonal antibody Clone #6G7, and an additional antibody heavy chain and light chain. In some embodiments, the bispecific antibody has a first heavy chain and a first light chain from monoclonal antibody Clone #6G10, and an additional antibody heavy chain and light chain.

TABLE 1
Anti-CD47 antibody amino acid sequences table.

| Clone # | Sequence names | SEQ ID NOs |
|---|---|---|
| Clone#6B8 | Heavy chain V domain | (SEQ ID NO: 1) |
| | Light chain V domain | (SEQ ID NO: 2) |
| Clone#6E12 | Heavy chain V domain | (SEQ ID NO: 17) |
| | Light chain V domain | (SEQ ID NO: 18) |
| Clone#6G7 | Heavy chain V domain | (SEQ ID NO: 33) |
| | Light chain V domain | (SEQ ID NO: 34) |
| Clone#6G10 | Heavy chain V domain | (SEQ ID NO: 49) |
| | Light chain V domain | (SEQ ID NO: 50) |
| Clone#1G4 | Heavy chain V domain | (SEQ ID NO: 65) |
| | Light chain V domain | (SEQ ID NO: 66) |

TABLE 2
Nucleic acid sequences encoding anti-
CD47 antibody variable domains.

| Clone # | Sequence names | SEQ ID NOs |
|---|---|---|
| Clone#6B8 | Heavy chain V domain | (SEQ ID NO: 9) |
| | Light chain V domain | (SEQ ID NO: 10) |
| Clone#6E12 | Heavy chain V domain | (SEQ ID NO: 25) |
| | Light chain V domain | (SEQ ID NO: 26) |
| Clone#6G7 | Heavy chain V domain | (SEQ ID NO: 41) |
| | Light chain V domain | (SEQ ID NO: 42) |
| Clone#6G10 | Heavy chain V domain | (SEQ ID NO: 57) |
| | Light chain V domain | (SEQ ID NO: 58) |
| Clone#1G4 | Heavy chain V domain | (SEQ ID NO: 73) |
| | Light chain V domain | (SEQ ID NO: 74) |

### Example 1. Anti-CD47 Antibody Production

The antibodies were created by electrofusion of human CD4740-immunized mouse spleenocytes (Balb/c strain) with SP2/0-Ag14 cells (ATCC). Splenocytes were collected from balb/c mice hyperimmunized with purchased recombinant human CD47 protein. Cell fusion was performed with the standard protocol from BTX. Fused cells were seeded into 96-well plates and beads-screening was conducted to identify antigen-bound magnetic beads that contain antibodies which interact with human CD47 protein. Positive wells were further expanded and follow a limited dilution to isolate monoclonal hybridomas. Purified antibodies were used to test their ability to bind CD47 and to neutralize interaction with SIRP-alpha.

### Example 2. Anti-CD47 Antibody Binding Assay

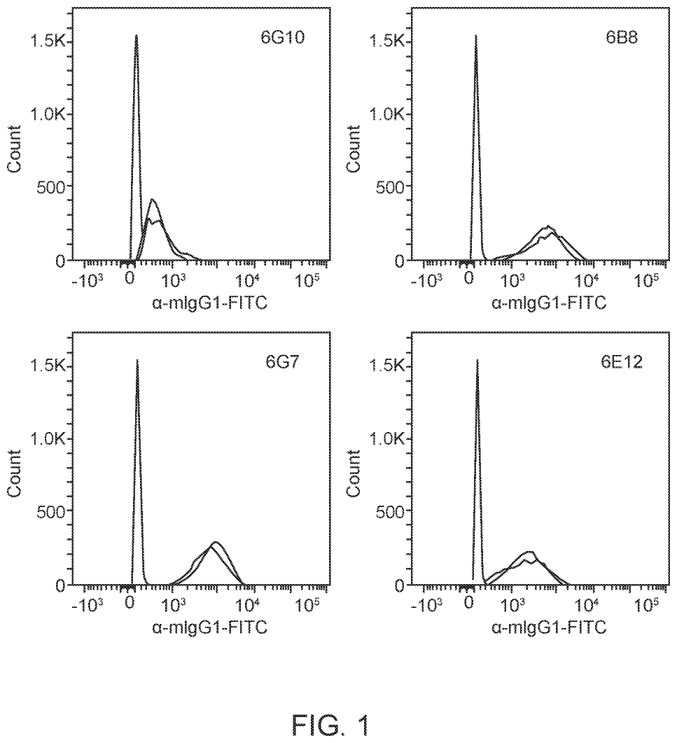

1×10⁶ H1299-hCD47 overexpression cells were stained by 1 μg AVI anti-hCD47 monoclonal antibody (clone 6G7, 6B8, 6G10, or 6E12) for 30 minutes. After washing, FITC-conjugated anti-mIgG1 (Jackson immunoresearch) was used as secondary antibody to detect mouse anti-hCD47 antibody on the cell surface. Flow cytometry was performed using Cytek NL-3000 and data was analyzed with FlowJo. Assay results shown in FIG. 1 demonstrate that these anti-hCD47 monoclonal antibodies bind human CD47 on the cell surface.

### Example 3. Anti-CD47 Antibody Binding Assay

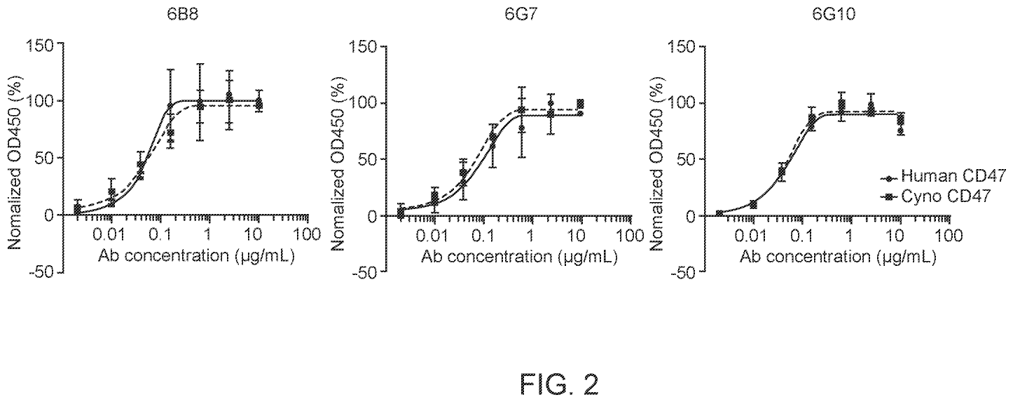

Human CD47-conjugated nanoparticles or cynomolgus CD47-conjugated nanoparticles were stained with various concentrations of AVI anti-hCD47 monoclonal antibody (clone 6G7, 6B8, 6G10) for 2 hours. After washing, HRP-conjugated anti-mIgG1 (Jackson immunoresearch) was used as secondary antibody to detect mouse anti-hCD47 antibody. TMB substrate solution and stop solution were added sequentially for single detection. Optical Density 450 nm was measured at by SpectraMax M2 Microplate Readers (Molecular Devices). Assay results shown in FIG. 2 demonstrate that these anti-hCD47 monoclonal antibodies bind human CD47 on the cell surface.

### Example 4. Anti-CD47 Antibody Binding Assay

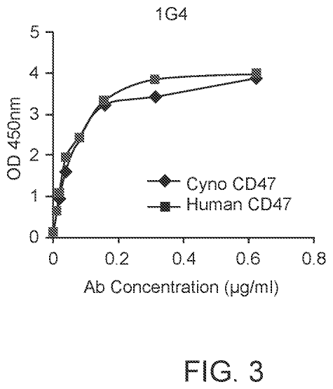

Human CD47-conjugated nanoparticles or cynomolgus CD47-conjugated nanoparticles were stained with various concentrations of AVI anti-hCD47 monoclonal antibody (clone 1G4) for 2 hours. After washing, HRP-conjugated anti-mIgG1 (Jackson immunoresearch) was used as secondary antibody to detect mouse anti-hCD47 antibody. TMB substrate solution and stop solution were added sequentially for single detection. Optical Density 450 nm was measured at by SpectraMax M2 Microplate Readers (Molecular Devices). Assay results shown in FIG. 3 demonstrate that clone 1G4 monoclonal antibodies bind both human and cynomolgus CD47.

### Example 5. Anti-CD47 Antibody Phagocytosis Assay

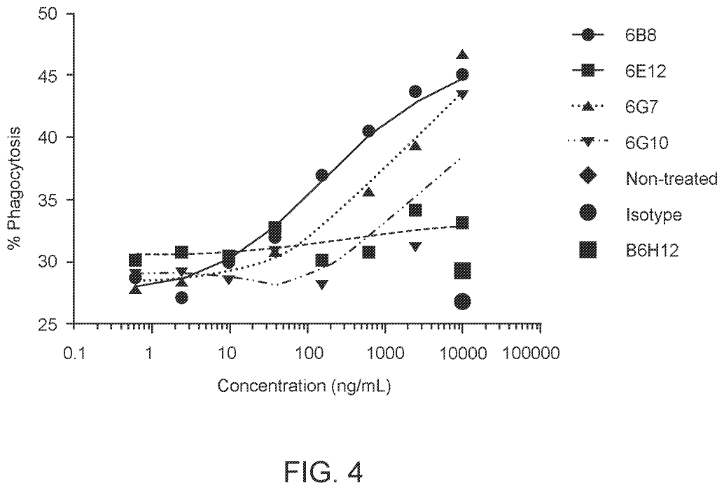

Monocytes were cultured in complete RPMI medium+10 ng/mL GM-CSF for 7 days to induce macrophage differentiation. Macrophages were future polarized to M1 macrophages by culturing 24 hours in complete RPMI medium+10-ng/mL GM-CSF +20-ng/mL IFN-γ+100-ng/mL LPS. Raji cells were labeled with 5-mM CFSE. M1 macrophages and CFSE-labeled Raji cells were co-cultured in 96 well plates with various concentrations of anti-hCD47 antibodies for 2 hours to initiate the process of phagocytosis. Fey receptors on Macrophages were blocked by Human TruStain FcX antibody (BioLegend). Macrophages were further stained with anti-CD11c, anti-CD45, anti-CD80 and PI (BioLegend). CD11c⁻/CD45⁺ were used as marker to identified macrophages. Phagocytosis index was measures with percentage of CFSE+ macrophages. Flow cytometry was performed using Cytek NL-3000 and data was analyzed with FlowJo. Assay results shown in FIG. 4 demonstrate that antibody clones 6B8, 6G7, 6G10, and 6E12 enhance the phagocytosis of macrophages.

### Example 6. Anti-CD47 Antibody Phagocytosis Assay

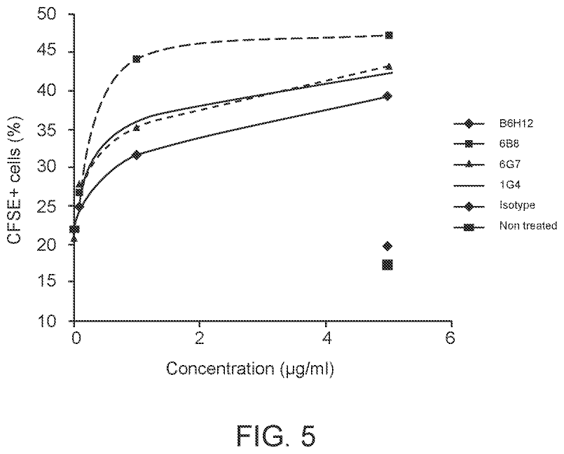

Monocytes were cultured in complete RPMI medium+10 ng/mL GM-CSF for 7 days to induce macrophage differentiation. Macrophages were future polarized to M1 macrophages by culturing 24 hours in complete RPMI medium+10-ng/mL GM-CSF +20-ng/mL IFN-γ+100-ng/mL LPS. Raji cells were labeled with 5-mM CFSE. M1 macrophages and CFSE-labeled Raji cells were co-cultured in 96 well plates with various concentrations of anti-hCD47 antibodies for 2 hours to initiate the process of phagocytosis. Fey receptors on Macrophages were blocked by Human TruStain FcX antibody (BioLegend). Macrophages were further stained with anti-CD11c, anti-CD45, anti-CD80 and PI (BioLegend). CD11c⁻/CD45⁺ were used as marker to identified macrophages. Phagocytosis index was measures with percentage of CFSE+ macrophages. Flow cytometry was performed using Cytek NL-3000 and data was analyzed with FlowJo. Assay results shown in FIG. 5 demonstrate that antibody clones 6B8, 6G7, and 1G4 enhance the phagocytosis of macrophages.

### Example 7. Anti-CD47 Antibody Binding Assay

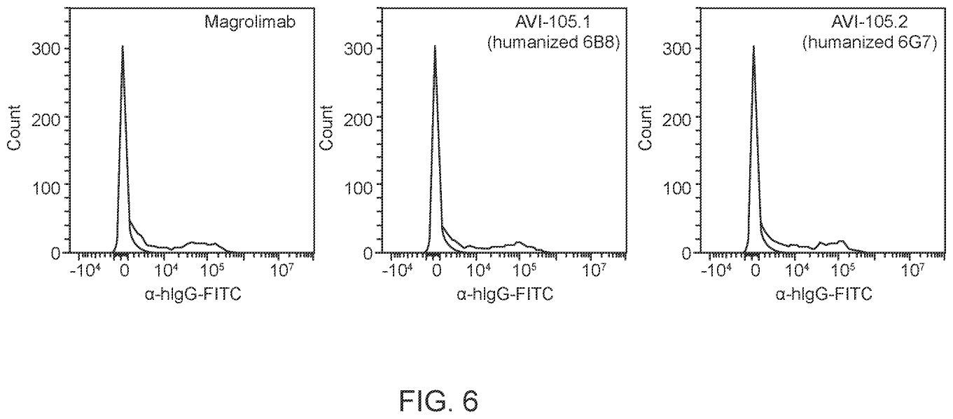

1×10⁶ H1299-hCD47 overexpression cells were stained by 1 μg AVI anti-hCD47 humanized mAbs (clone 6B8 or 6G7) for 30 minutes. After washing, FITC-conjugated anti-human IgG (Jackson immunoresearch) was used as secondary antibody to detect mouse anti-hCD47 antibody on the cell surface. Flow cytometry was performed using Cytek NL-3000 and data was analyzed with FlowJo. Assay results shown in FIG. 6 demonstrate that anti-hCD47 humanized mAbs (clone 6B8 and 6G7) retain binding ability to human CD47 on the cell surface.

### Example 8. Anti-CD47 Antibody In Vivo Anti-Tumor Assay

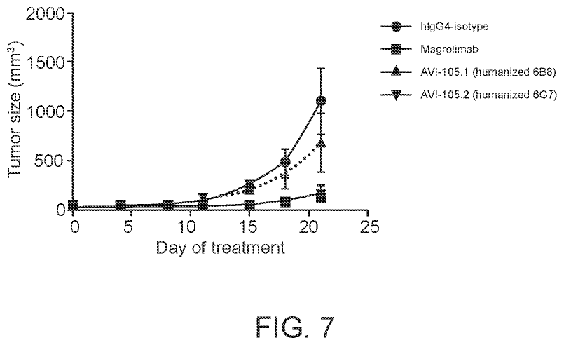

NCI-H82 (ATCC, 1.25×10⁶ cells/mouse) lung carcinoma cell line was used to establish xenograft tumors subcutaneously on 7 weeks old female NSG mice (The Jackson Laboratory). The antibody treatment started when tumor volume reached 50 mm³. Mice were treated three times weekly with 10 mg/kg antibody. The antibody was administrated by intraperitoneal injection for 3 weeks. Tumor size will be measured two times weekly after treatment started in two dimensions using a caliper, and the volume is expressed in mm³ using the formula: V=0.5 a×b² where a and b are the long and short dimensions of the tumor, respectively. Assay results shown in FIG. 7 demonstrate that humanized clones 6B8 and 6G7 had in vivo anti-tumor efficacy.

Although the above invention has been described in some detail by way of illustration and example for purposes of clarity of understanding, it is apparent to those skilled in the art that certain changes and modifications will be practiced. Therefore, the description and examples should not be construed as limiting the scope of the disclosed invention.

The disclosures of all publications, patents, patent applications and published patent applications referred to herein by an identifying citation are hereby incorporated herein by reference in their entirety. All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The sequence listing of the polynucleotide/peptide sequences described herein is included in the Appendix hereto, which is incorporated herein by reference in its entirety.

### REFERENCES

Advani, et al., “CD47 Blockade by Hu5F9-G4 and Rituximab in Non-Hodgkin's Lymphoma”. N Engl J Med. 2018 Nov. 1; 379(18):1711-1721.
Bird, et al., “Single-chain antigen-binding proteins”. Science. 1988 October, 242(4877):423-6.
Gennaro, ed., “Remington: The Science and Practice of Pharmacy”. 20th Edition. Lippincott Williams & Wilkins. 2003.
Huston, et al., Protein engineering of antibody binding sites: recovery of specific activity in an anti-digoxin single-chain Fv analogue produced in Escherichia coli”. Proc Natl Acad Sci USA. 1988 August; 85(16): 5879-5883.
J. R. Robinson ed., “Sustained and Controlled Release Drug Delivery Systems”. Marcel Dekker, Inc., New York, 1978.
Jones et al., “Replacing the complementarity-determining regions in a human antibody with those from a mouse”. Nature. 1986; 4,321(6069):522-5.
Kabat et al., “Sequences of Proteins of Immunological Interest”, Fifth Edition, U.S. Department of Health and Human Services, 1991; NIH Publication No. 91-3242
Kostelny et al., “Formation of a bispecific antibody by the use of leucine zippers”. J Immunol. 1992 Mar. 1; 148(5):1547-53.
Mahoney et al., 2015. Combination cancer immunotherapy and new immunomodulatory targets. Nat Rev Drug Discov 14: 561-584.
Paul et al., “Fundamental immunology”. Philadelphia: Wolters Kluwer Health/Lippincott Williams & Wilkins, 2013.
Postow, et al., 2015, “Nivolumab and ipilimumab versus ipilimumab in untreated melanoma.” N Engl J Med. 2015; 372(21):2006-17.
Queens et al., “A humanized antibody that binds to the interleukin 2 receptor”. Proc Natl Acad Sci USA. 1989 December; 86(24): 10029-10033.
Sambrook J et al., “Molecular Cloning: A Laboratory Manual”, Third Edition, 2000.
Songsivilai & Lachmarm, “Bispecific antibody: a tool for diagnosis and treatment of disease”. Clin Exp Immunol. 1990 March; 79(3): 315-321.
Wolchok et al., Nivolumab plus ipilimumab in advanced melanoma. N Engl J Med 2013; 369:122-33.
“Yervoy, ipilimumab (BMS-734016)—Product Profile—BioCentury”. BioCentury Online Intelligence. BioCentury Publications. Retrieved 11 Aug. 2016.

Sequence Listing
Clone#6B8
Amino Acids
Heavy chain:
(SEQ ID NO: 1)
VQLQQSGPEMVKPGASVKMSCKASGYTFTNYVLHWVKQKPGQGLEWIGYINP
YNDGTNYNAKFKDKATLTSDKSSSTAYMELSSLTSEDSAVYYCSKGGYYTLDY
WGQGTSVTVSS
Light chain:
(SEQ ID NO: 2)
DVVMTQTPLSLPVSFGDQASISCRSSQSLVHSKGNTYLHWYLQKPGQSPKLLIY
KISNRFSGVPDRFSGSGSGTDFTLKISRVEAEDLGVYFCSQSTHVPFTFGSGTKLE
IK
Heavy Chain CDRs
CDR1
(SEQ ID NO: 3)
GYTFTNYV
CDR2
(SEQ ID NO: 4)
INPYNDGT
CDR3
(SEQ ID NO: 5)
SKGGYYTLDY
Light Chain CDRs
CDR1
(SEQ ID NO: 6)
QSLVHSKGNTY
CDR2
(SEQ ID NO: 7)
KIS
CDR3
(SEQ ID NO: 8)
SQSTHVPFT
Nucleotide:
Heavy chain:
(SEQ ID NO: 9)
GTG CAA CTC CAA CAA TCA GGA CCG GAA ATG GTG AAA CCG GGC GCA
TCC GTC AAA ATG TCA TGC AAG GCC AGC GGC TAC ACC TTT ACT AAT
TAC GTT CTC CAT TGG GTA AAA CAA AAA CCA GGC CAA GGA CTG GAA
TGG ATA GGG TAT ATA AAT CCA TAT AAC GAC GGG ACT AAC TAC AAT
GCA AAA TTC AAA GAT AAA GCT ACA CTG ACG AGT GAT AAG TCA TCT TCT
ACC GCC TAC ATG GAG TTG AGC TCT CTC ACT TCA GAG GAC TCC GCT GTC
TAC TAT TGT TCT AAG GGA GGA TAC TAT ACA CTC GAT TAC TGG GGG
CAG GGT ACG TCT GTT ACC GTT TCA AGC
Light chain:
(SEQ ID NO: 10)
GAT GTC GTG ATG ACG CAA ACA CCC CTG TCC CTG CCA GTT TCT TTT
GGA GAT CAA GCA TCT ATC AGT TGC CGC TCT AGT CAA TCT CTT GTT
CAC AGT AAA GGC AAC ACC TAC CTC CAT TGG TAC TTG CAA AAA CCA
GGC CAG TCC CCT AAA TTG CTT ATC TAC AAA ATA AGT AAC AGG TTT
AGC GGT GTG CCT GAC AGA TTT TCT GGG TCA GGA TCT GGC ACA GAC
TTT ACA CTC AAA ATC AGT CGG GTG GAA GCC GAG GAT CTC GGG GTG
TAC TTT TGC AGT CAG TCT ACT CAC GTT CCA TTC ACT TTT GGG TCA
GGC ACG AAG CTT GAG ATT AAG
Heavy Chain CDRs
CDR1
(SEQ ID NO: 11)
GGC TAC ACC TTT ACT AAT TAC GTT
CDR2
(SEQ ID NO: 12)
ATA AAT CCA TAT AAC GAC GGG ACT
CDR3
(SEQ ID NO: 13)
TCT AAG GGA GGA TAC TAT ACA CTC GAT TAC
Light Chain CDRs
CDR1
(SEQ ID NO: 14)
CAA TCT CTT GTT CAC AGT AAA GGC AAC ACC TAC
CDR2
(SEQ ID NO: 15)
AAA ATC AGT
CDR3
(SEQ ID NO: 16)
AGT CAG TCT ACT CAC GTT CCA TTC ACT
Clone#6E12
Amino Acids
Heavy chain:
(SEQ ID NO: 17)
QVQLQQSGAELVKPGASVKLSCKASGNTFINYYVYWVKQRPGQGLEWIGEINP
SSGSANFNAEFKSKATLTVDKSSSTAYIQLSSLTSEDSAVYYCINGYFGTYWGQ
GTLVTVSA
Light chain:
(SEQ ID NO: 18)
DIQMTQSPASLSASVGETVTISCRASENVYSYLAWYQQKQGKSPQLLVYSANTL
AEGVPSRFSGSGSGTRFSLKINSLQSEDFGSYYCQHHYGSPSAFGGGTKLEI
Heavy Chain CDRs
CDR1
(SEQ ID NO: 19)
GNTFINYY
CDR2
(SEQ ID NO: 20)
INPSSGSA
CDR3
(SEQ ID NO: 21)
INGYFGTY
Light Chain CDRs
CDR1
(SEQ ID NO: 22)
ENVYSY
CDR2
(SEQ ID NO: 23)
SAN
CDR3
(SEQ ID NO: 24)
QHHYGSPSA
Nucleotide:
Heavy chain:
(SEQ ID NO: 25)
CAG GTT CAA CTC CAA CAA TCC GGT GCA GAA TTG GTA AAG CCA GGC
GCG TCT GTA AAG CTG AGC TGC AAA GCC AGC GGT AAT ACG TTC ATC
AAT TAC TAC GTA TAT TGG GTT AAA CAG CGC CCA GGC CAA GGA CTC
GAA TGG ATC GGC GAA ATA AAT CCC AGC TCT GGA AGT GCA AAC TTT
AAC GCT GAG TTT AAG TCC AAA GCC ACG CTG ACC GTT GAC AAG TCT
AGT AGC ACC GCA TAC ATC CAA CTT AGT TCC CTG ACC AGT GAG GAC
AGC GCC GTT TAT TAT TGC ATT AAC GGC TAT TTC GGG ACA TAC TGG
GGC CAG GGG ACT TTG GTG ACT GTA AGC GCG
Light chain:
(SEQ ID NO: 26)
GAC ATT CAG ATG ACC CAA TCC CCT GCC AGC CTT TCA GCC TCC GTG
GGT GAA ACT GTT ACT ATT AGC TGT AGA GCT TCA GAG AAT GTG TAT
TCT TAC TTG GCA TGG TAT CAA CAG AAG CAG GGA AAG TCA CCC CAA
CTC TTG GTA TAT AGC GCC AAT ACC CTG GCG GAA GGG GTC CCA TCT
CGA TTT TCC GGG TCT GGG AGC GGG ACT AGA TTT TCC CTC AAG ATC
AAC TCT CTT CAG AGT GAG GAC TTT GGT TCC TAT TAT TGC CAA CAT
CAC TAT GGT TCA CCT AGT GCG TTC GGG GGT GGA ACC AAG TTG GAG
ATA
Heavy Chain CDRs
CDR1
(SEQ ID NO: 27)
GGT AAT ACG TTC ATC AAT TAC TAC
CDR2
(SEQ ID NO: 28)
ATA AAT CCC AGC TCT GGA AGT GCA
CDR3
(SEQ ID NO: 29)
ATT AAC GGC TAT TTC GGG ACA TAC
Light Chain CDRs
CDR1
(SEQ ID NO: 30)
GAG AAT GTG TAT TCT TAC
CDR2
(SEQ ID NO: 31)
AGC GCC AAT
CDR3
(SEQ ID NO: 32)
CAA CAT CAC TAT GGT TCA CCT AGT GCG
Clone#6G7
Amino Acids
Heavy chain:
(SEQ ID NO: 33)
VKLVESGGGLVKPGGSLKLSCAASGFAFSTYDMSWIRQTPEKRLEWVATISTGG
TYTYYPDSVKGRFTISRDNARNTLYLQMSSLRSEDTALYYCSRRPYYFDYWGQ
GTTLTVSS
Light chain:
(SEQ ID NO: 34)
DIKMTQSPSSMYASLGERVTITCKASQDINSYLSWFQQKPGKSPKTLIYRVNRLV
DGVPSRFSGSGSGQDYSLTISSLEYEDMGIYYCLQYDEFPLTFGAGTKLELK
Heavy Chain CDRs
CDR1
(SEQ ID NO: 35)
GFAFSTYD
CDR2
(SEQ ID NO: 36)
ISTGGTYT
CDR3
(SEQ ID NO: 37)
SRRPYYFDY
Light Chain CDRs
CDR1
(SEQ ID NO: 38)
QDINSY
CDR2
(SEQ ID NO: 39)
RVN
CDR3
(SEQ ID NO: 40)
LQYDEFPLT
Nucleotide:
Heavy chain:
(SEQ ID NO: 41)
GTT AAG CTG GTG GAA AGT GGA GGA GGC TTG GTC AAA CCG GGG GGG
TCC CTT AAA CTC AGT TGT GCG GCC AGC GGC TTT GCT TTC AGC ACT
TAC GAT ATG AGC TGG ATC CGC CAG ACG CCA GAG AAG AGG CTT GAG
TGG GTA GCA ACT ATT AGT ACC GGT GGG ACT TAT ACC TAC TAT CCG
GAT TCC GTG AAA GGA CGC TTC ACG ATT TCC CGC GAT AAC GCA CGC
AAT ACT CTG TAT CTG CAA ATG TCT TCT TTG AGA AGT GAG GAC ACA
GCC TTG TAC TAC TGC AGT CGC CGC CCA TAC TAT TTC GAC TAC TGG
GGC CAG GGC ACG ACC CTG ACG GTG TCA AGT
Light chain:
(SEQ ID NO: 42)
GAC ATA AAA ATG ACC CAA TCC CCG TCT TCA ATG TAC GCA AGC CTT
GGG GAG CGA GTG ACG ATC ACC TGC AAG GCA AGC CAA GAC ATT AAC
TCC TAT CTT TCA TGG TTT CAG CAA AAA CCC GGC AAG AGT CCG AAA
ACC CTG ATA TAT AGA GTG AAC CGC CTT GTG GAT GGG GTC CCA AGC
CGC TTC TCT GGA TCT GGT TCT GGC CAG GAC TAC TCC CTC ACG ATT TCA
TCC CTC GAA TAT GAA GAC ATG GGA ATC TAC TAT TGT CTC CAA TAC
GAC GAG TTT CCT CTG ACG TTT GGG GCG GGG ACC AAA TTG GAA CTT
AAA
Heavy Chain CDRs
CDR1
(SEQ ID NO: 43)
GGC TTT GCT TTC AGC ACT TAC
CDR2
(SEQ ID NO: 44)
ATT AGT ACC GGT GGG ACT TAT ACC
CDR3
(SEQ ID NO: 45)
AGT CGC CGC CCA TAC TAT TTC GAC TAC
Light Chain CDRs
CDR1
(SEQ ID NO: 46)
CAA GAC ATT AAC TCC TAT
CDR2
(SEQ ID NO: 47)
AGA GTG AAC
CDR3
(SEQ ID NO: 48)
CTC CAA TAC GAC GAG TTT CCT CTG ACG
Clone#6G10
Amino Acids
Heavy chain:
(SEQ ID NO: 49)
VQLQQSGAEVVRPGTSVKMSCKAAGYTFTNYWIGWVKQRPGHGLEWIGDISP
GGDYSNYNEKFKGKATLTADTSSSTAYMQLSSLTSEDSAIYYCARKGKGGMDS
WGQGTSVTVSS
Light chain:
(SEQ ID NO: 50)
DVLMTQTPLSLPVSLGDQASISCRSSQSIVHSNGNTYLEWYLQKPGQSPKLLIYK
VSNRFSGVPDRFSGSGSGTDFTLKISRVEAEDLGVYYCFQGSHVPWTFGGGTKL
EIK
Heavy Chain CDRs
CDR1
(SEQ ID NO: 51)
GYTFTNYW
CDR2
(SEQ ID NO: 52)
ISPGGDYS
CDR3
(SEQ ID NO: 53)
ARKGKGGMDS
Light Chain CDRs
CDR1
(SEQ ID NO: 54)
QSIVHSNGNTY
CDR2
(SEQ ID NO: 55)
KVS
CDR3
(SEQ ID NO: 56)
FQGSHVPWT
Nucleotide:
Heavy chain:
(SEQ ID NO: 57)
GTA CAG CTT CAA CAA TCC GGG GCC GAG GTG GTA AGA CCC GGA ACA
TCT GTT AAA ATG TCT TGC AAG GCC GCC GGG TAC ACG TTC ACG AAC
TAT TGG ATT GGG TGG GTC AAA CAA AGG CCA GGG CAT GGC TTG GAG
TGG ATA GGA GAC ATT TCC CCT GGA GGC GAT TAC AGC AAT TAC AAC
GAA AAG TTC AAA GGA AAG GCA ACA TTG ACC GCG GAC ACA AGT AGT
TCT ACA GCT TAT ATG CAA CTC AGC TCC CTG ACC TCT GAG GAT TCT GCC
ATA TAC TAC TGC GCT CGG AAG GGA AAG GGC GGA ATG GAC TCA TGG
GGT CAG GGA ACA AGT GTT ACG GTC TCT AGC
Light chain:
(SEQ ID NO: 58)
GAC GTT CTG ATG ACT CAG ACC CCA TTG AGC CTG CCT GTT TCT CTC
GGA GAT CAA GCG TCA ATT AGC TGT CGC AGT TCT CAG TCT ATT GTG
CAT AGC AAC GGC AAC ACA TAT CTG GAA TGG TAT TTG CAA AAA CCC
GGC CAG TCC CCT AAA TTG CTC ATA TAT AAG GTC AGC AAT CGC TTC
AGT GGG GTG CCA GAC CGA TTC TCA GGC AGC GGT TCT GGT ACA GAC
TTC ACA CTG AAG ATA AGC CGA GTA GAA GCT GAG GAC TTG GGG GTG
TAC TAT TGC TTC CAA GGC TCC CAC GTT CCT TGG ACT TTC GGA GGT
GGA ACT AAG TTG GAG ATA AAG
Heavy Chain CDRs
CDR1
(SEQ ID NO: 59)
GGG TAC ACG TTC ACG AAC TAT TGG
CDR2
(SEQ ID NO: 60)
ATT TCC CCT GGA GGC GAT TAC AGC
CDR3
(SEQ ID NO: 61)
GCT CGG AAG GGA AAG GGC GGA ATG GAC TCA
Light Chain CDRs
CDR1
(SEQ ID NO: 62)
CAG TCT ATT GTG CAT AGC AAC GGC AAC ACA TAT
CDR2
(SEQ ID NO: 63)
AAG GTC AGC
CDR3
(SEQ ID NO: 64)
TTC CAA GGC TCC CAC GTT CCT TGG ACT
Clone#1G4
Amino acid sequence
Heavy chain variable domain amino acid sequence (136 aa)
Signal peptide-FR1-CDR1-FR2-CDR2-FR3-CDR3-FR4
(SEQ ID NO: 65)
MEWSWIFLFLLSGTAGVHSEVQLQQSGPEMVKPGASVKMSCKASGYTFTNYVL
HWVKQKPGQGLEWIGYINPYNDGTKYNAKFKGKATLTSDKSSSTAYMELSSLT
SEDSAVYYCTKGGYYTLDYWGQGTSVTVSS
Light chain variable domain amino acid sequence (131 aa)
Signal peptide-FR1-CDR1-FR2-CDR2-FR3-CDR3-FR4
(SEQ ID NO: 66)
MKLPVRLLVLMFWIPASSSDVVMTQTPLSLPVSFGDQASISCRSSQSLVHSKGNT
YLHWYLQKPGQSPKLLIYKISNRFSGVPDRFSGSGSGTDFTLKISRVEAEDLGVY
FCSQSTHVPFTFGSGTKLEIK
Heavy Chain CDRs
CDR1
(SEQ ID NO: 67)
NYVLH
CDR2
(SEQ ID NO: 68)
YINPYNDGTKYNAKFKG
CDR3
(SEQ ID NO: 69)
GGYYTLDY
Light Chain CDRs
CDR1
(SEQ ID NO: 70)
RSSQSLVHSKGNTYLH
CDR2
(SEQ ID NO: 71)
KISNRFS
CDR3
(SEQ ID NO: 72)
SQSTHVPFT
Nucleotide:
Heavy chain DNA sequence (408 bp)
Signal sequence-FR1-CDR1-FR2-CDR2-FR3-CDR3-FR4
(SEQ ID NO: 73)
ATG GAA TGG AGT TGG ATA TTT CTC TTT CTC CTG TCA GGA ACT GCA
GGT GTC CAC TCT GAG GTC CAG CTG CAG CAG TCT GGA CCT GAG ATG
GTA AAG CCT GGG GCT TCA GTG AAG ATG TCC TGC AAG GCT TCT GGA
TAC ACA TTC ACT AAC TAT GTT TTG CAC TGG GTG AAG CAG AAG CCT
GGG CAG GGC CTT GAG TGG ATT GGA TAT ATT AAT CCT TAC AAT GAT
GGT ACT AAG TAC AAT GCG AAG TTC AAA GGC AAG GCC ACA CTG ACT
TCA GAC AAA TCC TCC AGC ACA GCC TAC ATG GAG CTC AGC AGC CTG
ACC TCT GAG GAC TCT GCG GTC TAT TAC TGT ACA AAG GGC GGT TAC
TAT ACT CTG GAC TAC TGG GGT CAA GGG ACC TCA GTC ACC GTC TCC
TCA
Light chain DNA sequence (393 bp) Signal
sequence-FR1-CDR1-FR2-CDR2-FR3-CDR3-FR4
(SEQ ID NO: 74)
ATG AAG TTG CCT GTT AGG CTG TTG GTG CTG ATG TTC TGG ATT CCT GCT
TCC AGC AGT GAT GTT GTG ATG ACC CAA ACT CCA CTC TCC CTG CCT
GTC AGT TTT GGA GAT CAA GCC TCC ATC TCT TGC AGA TCT AGT CAG
AGC CTT GTA CAC AGT AAG GGA AAC ACC TAT TTA CAT TGG TAC CTG
CAG AAG CCA GGC CAG TCT CCA AAG CTC CTG ATT TAC AAA ATC TCC
AAC CGA TTT TCT GGG GTC CCA GAC AGG TTC AGT GGC AGT GGA TCA
GGG ACA GAT TTC ACA CTC AAG ATC AGC AGA GTG GAG GCT GAG GAT
CTG GGA GTT TAT TTC TGC TCT CAA AGT ACA CAT GTT CCA TTC ACG
TTC GGC TCG GGG ACA AAG TTG GAA ATA AAA
Heavy Chain CDRs
CDR1
(SEQ ID NO: 75)
AAC TAT GTT TTG CAC
CDR2
(SEQ ID NO: 76)
TAT ATT AAT CCT TAC AAT GAT GGT ACT AAG TAC AAT GCG AAG TTC
AAA GGC
CDR3
(SEQ ID NO: 77)
GGC GGT TAC TAT ACT CTG GAC TAC
Light Chain CDRs
CDR1
(SEQ ID NO: 78)
AGA TCT AGT CAG AGC CTT GTA CAC AGT AAG GGA AAC ACC TAT TTA
CAT
CDR2
(SEQ ID NO: 79)
AAA ATC TCC AAC CGA TTT TCT
CDR3
(SEQ ID NO: 80)
TCT CAA AGT ACA CAT GTT CCA TTC ACG

We claim:

1. An isolated monoclonal antibody, or antigen-binding portion thereof, comprising
a heavy chain variable region CDR1 comprising SEQ ID NO:3;
a heavy chain variable region CDR2 comprising SEQ ID NO:4;
a heavy chain variable region CDR3 comprising SEQ ID NO:5;
a light chain variable region CDR1 comprising SEQ ID NO:6;
a light chain variable region CDR2 comprising SEQ ID NO:7; and
a light chain variable region CDR3 comprising SEQ ID NO:8,
wherein said antibody or portion specifically binds to human CD47.

2. An isolated monoclonal antibody, or antigen-binding portion thereof, comprising
a heavy chain variable region CDR1 comprising SEQ ID NO:19;
a heavy chain variable region CDR2 comprising SEQ ID NO:20;
a heavy chain variable region CDR3 comprising SEQ ID NO:21;
a light chain variable region CDR1 comprising SEQ ID NO:22;
a light chain variable region CDR2 comprising SEQ ID NO:23; and
a light chain variable region CDR3 comprising SEQ ID NO:24,
wherein said antibody or portion specifically binds to human CD47.

3. An isolated monoclonal antibody, or antigen-binding portion thereof, comprising:
a heavy chain variable region CDR1 comprising SEQ ID NO:35;
a heavy chain variable region CDR2 comprising SEQ ID NO:36;
a heavy chain variable region CDR3 comprising SEQ ID NO:37;
a light chain variable region CDR1 comprising SEQ ID NO:38;
a light chain variable region CDR2 comprising SEQ ID NO:39; and
a light chain variable region CDR3 comprising SEQ ID NO:40,
wherein said antibody or portion specifically binds to human CD47.

4. An isolated monoclonal antibody, or antigen-binding portion thereof, comprising:
a heavy chain variable region CDR1 comprising SEQ ID NO:51;
a heavy chain variable region CDR2 comprising SEQ ID NO:52;
a heavy chain variable region CDR3 comprising SEQ ID NO:53;
a light chain variable region CDR1 comprising SEQ ID NO:54;
a light chain variable region CDR2 comprising SEQ ID NO:55; and
a light chain variable region CDR3 comprising SEQ ID NO:56,
wherein said antibody or portion specifically binds to human CD47.

5. An isolated monoclonal antibody, or antigen-binding portion thereof, comprising:
a heavy chain variable region CDR1 comprising SEQ ID NO:67;
a heavy chain variable region CDR2 comprising SEQ ID NO:68;
a heavy chain variable region CDR3 comprising SEQ ID NO:69;
a light chain variable region CDR1 comprising SEQ ID NO:70;
a light chain variable region CDR2 comprising SEQ ID NO:71; and
a light chain variable region CDR3 comprising SEQ ID NO:72,
wherein said antibody or portion specifically binds to human CD47.

6. The antibody or antigen-binding portion according to any one of claims 1-5 that comprises a Fab fragment, an F(ab′)₂ fragment, an Fv fragment, a single chain antibody, or a bispecific antibody.

7. A pharmaceutical composition comprising the antibody or antigen-binding portion-according to any one of claims 1-5 and a pharmaceutically acceptable carrier.

8. A method of treating cancer in a human, comprising the step of administering to the human the antibody or antigen-binding portion according to any one of claims 1-5 in an amount effective to treat said cancer.

9. A method of enhancing an immune response in a subject in need thereof, comprising the step of administering to the subject an effective amount of the antibody or antigen-binding portion according to any one of claims 1-5, wherein said response is indicated by inhibition of the CD47 signaling that contributes to innate immunity.

10. The monoclonal antibody, or an antigen-binding portion thereof of any one of claims 1-5, wherein the antibody stimulates an anti-tumor immune response.

11. An isolated monoclonal antibody, or antigen-binding portion thereof, comprising:
a heavy chain variable region comprising an amino acid sequence selected from the group consisting of SEQ ID NOs: 1, 17, 33, 49, and 65; and
a light chain variable region comprising an amino acid sequence selected from the group consisting of SEQ ID NOs: 2, 18, 34, 50, and 66; wherein said antibody or portion specifically binds to human CD47.

12. A monoclonal antibody or an antigen-binding portion thereof according to claim 10, wherein the heavy chain variable domain comprising SEQ ID NO:1 and the light chain variable domain comprising SEQ ID NO:2.

13. A monoclonal antibody or an antigen-binding portion thereof according to claim 10, wherein the heavy chain variable domain comprising SEQ ID NO:17 and the light chain variable domain comprising SEQ ID NO:18.

14. A monoclonal antibody or an antigen-binding portion thereof according to claim 10, wherein the heavy chain variable domain comprising SEQ ID NO:33 and the light chain variable domain comprising SEQ ID NO:34.

15. A monoclonal antibody or an antigen-binding portion thereof according to claim 10, wherein the heavy chain variable domain comprising SEQ ID NO:49 and the light chain variable domain comprising SEQ ID NO:50.

16. A monoclonal antibody or an antigen-binding portion thereof according to claim 10, wherein the heavy chain variable domain comprising SEQ ID NO:65 and the light chain variable domain comprising SEQ ID NO:66.

17. The antibody or antigen-binding portion according to any one of claims 11-16 that comprises a Fab fragment, an F(ab′)₂ fragment, an Fv fragment, a single chain antibody, or a bispecific antibody.

18. A pharmaceutical composition comprising the antibody or antigen-binding portion-according to any one of claims 11-16 and a pharmaceutically acceptable carrier.

19. A method of treating cancer in a human, comprising the step of administering to the human the antibody or antigen-binding portion according to any one of claims 11-16 in an amount effective to treat said cancer.

20. A method of enhancing an immune response in a subject in need thereof, comprising the step of administering to the subject an effective amount of the antibody or antigen-binding portion according to any one of claims 11-16, wherein said response is indicated by activation of antigen presenting cells in the subject in need thereof.

21. The antibody of any one of claims 11-16, wherein the antibody stimulates an anti-tumor immune response.

22. The antibody according to any one of claims 11-16, that comprises an immunoglobulin G (IgG), an IgM, an IgE, an IgA or an IgD molecule.

23. The monoclonal antibody according to claim 22, wherein said antibody comprises a chimeric antibody or humanized antibody.

24. The monoclonal antibody according to claim 22, wherein said antibody comprises an IgG1, IgG2, IgG3, or IgG4.

25. A monoclonal antibody or an antigen-binding portion thereof according to claim 11, wherein said antibody comprises: a heavy chain variable domain amino acid sequence encoded by a nucleic acid sequence comprising SEQ ID NO:9 and a light chain variable domain amino acid sequence encoded by a nucleic acid sequence comprising SEQ ID NO:10.

26. A monoclonal antibody or an antigen-binding portion thereof according to claim 11, wherein said antibody comprises: a heavy chain variable domain amino acid sequence encoded by a nucleic acid sequence comprising SEQ ID NO:25 and a light chain variable domain amino acid sequence encoded by a nucleic acid sequence comprising SEQ ID NO:26.

27. A monoclonal antibody or an antigen-binding portion thereof according to claim 11, wherein said antibody comprises: a heavy chain variable domain amino acid sequence encoded by a nucleic acid sequence comprising SEQ ID NO:41 and a light chain variable domain amino acid sequence encoded by a nucleic acid sequence comprising SEQ ID NO:42.

28. A monoclonal antibody or an antigen-binding portion thereof according to claim 11, wherein said antibody comprises: a heavy chain variable domain amino acid sequence encoded by a nucleic acid sequence comprising SEQ ID NO:57 and a light chain variable domain amino acid sequence encoded by a nucleic acid sequence comprising SEQ ID NO:58.

29. A monoclonal antibody or an antigen-binding portion thereof according to claim 11, wherein said antibody comprises: a heavy chain variable domain amino acid sequence encoded by a nucleic acid sequence comprising SEQ ID NO:73 and a light chain variable domain amino acid sequence encoded by a nucleic acid sequence comprising SEQ ID NO:74.

30. An immunoconjugate comprising the antibody or portion according to any one of claims 1-5.

31. A bispecific antibody comprising the antibody or portion according to any one of claims 1-5.

32. The monoclonal antibody or antigen-binding portion in any one of claims 1-5 comprising at least two-F(ab) regions capable of specific binding to human CD47 and inhibits CD47 signaling thereby contributing to innate immunity.

33. A bispecific or multi-specific antibody comprising an antibody or portion binding to CTLA-4 and the antibody or portion according to any one of claims 1-5.

34. A bispecific or multi-specific antibody comprising an antibody or portion binding to CD40 and the antibody or portion according to any one of claims 1-5.

35. An immunoconjugate comprising a CpG oligodeoxynucleotide and the antibody or portion according to any one of claims 1-5.
